(19) United States
(12) Patent Application Publication
(10) Pub. No.: US 2004/0158261 A1
(43) Pub. Date: Aug. 12, 2004

(54) Endoscopic device for spill-proof laparoscopic ovarian cystectomy

(76) Inventor: Dinh Q. Vu, Palo Alto, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/770,285

(22) Filed: Feb. 2, 2004

Related U.S. Application Data
(63) Continuation-in-part of application No. 10/147,261, filed on May 15, 2002, now Pat. No. 6,685,628.

(51) Int. Cl. A61B 17/22

(52) U.S. Cl. 606/114; 606/127; 600/37; 600/207

(57) ABSTRACT

A endoscopic device for use in a laparoscopic surgical procedure such as a cystectomy is disclosed. An intact organ such as an ovary is encapsulated partially or completely with the endoscopic device while the organ remains attached to the body. By inserting instruments into the endoscopic device, a surgical procedure is then performed within the endoscopic device, for example, a cyst may be removed from an ovary. After cyst removal, the endoscopic device is irrigated to remove leakage before withdrawal of the cyst within the endoscopic device.

### CROSS-REFERENCE TO RELATED APPLICATION

This application is a continuation-in-part of U.S. application Ser. No. 10/147,261, filed May 15, 2002, the entirety of which is hereby incorporated by reference.

### BACKGROUND OF THE INVENTION

1. Field of the Invention

The invention relates in one embodiment to a method of performing an ovarian cystectomy by laparoscopy using a spill-proof endoscopic device. A spill-proof endoscopic device is also disclosed.

1. Description of the Related Art

Within the context of one embodiment of the present invention, ovarian cystectomy refers to the removal of a cyst from an ovary. Laparotomy refers to the traditional method of opening up the abdomen by making a large incision, typically about 12-14 cm in length. Oophorectomy is defined as the removal of the entire ovary. Laparoscopy is a method of performing intra-abdominal surgery via very small incisions, often 1 cm or less in length. Laparoscopic ovarian cystectomy is the removal of a cyst from the ovary using the technique of laparoscopy.

Ovarian cystectomy is a commonly performed procedure in gynecology. An attendant risk to this procedure, especially if done via laparoscopy, is the accidental leakage of the cyst content into the peritoneal cavity, which occurs most often during the separation of the cyst from the ovary. This can worsen the prognosis if the cyst turns out to be malignant or can lead to peritonitis if the cyst is a dermoid or mucinous cyst. Because of this hazard, ovarian cystectomy by laparoscopy requires more skill, time, and effort than that done via laparotomy if the cyst is to be removed without spillage. Consequently, many surgeons will opt to go directly to a laparotomy or perform a laparoscopic oophorectomy (which has a much lower risk of cyst leakage because the cyst is not separated from the ovary) instead of a laparoscopic cystectomy if he or she is uncertain about the nature of the cyst.

In addition, there is often no way to know in advance if the cyst is malignant, dermoid, mucinous, or otherwise. The physician must rely upon a combination of imprecise diagnostic indicators and guess work to determine whether or not a laparoscopic cystectomy can be safely attempted. This uncertainty and the fear of spillage cause many physicians not to attempt laparoscopic ovarian cystectomy or to feel very uncomfortable doing it.

Thus, a need exists for methods and apparatuses for performing a laparoscopic ovarian cystectomy safely and easily, without spilling the contents of the cyst into the peritoneal cavity. This will reduce the number of patients who suffer the harmful consequences of cyst spillage. This will also allow more laparoscopic ovarian cystectomies, instead of laparotomies or oophorectomies, to be performed.

The prior art discloses several types of endo-bags for use during a laparoscopic procedure. U.S. Pat. No. 5,215,521 to Cochran et al. discloses an entrapment envelope which is inserted through a laparoscopic port for removal of a surgically severed organ.

U.S. Pat. No. 6,059,793 to Pagedas discloses a reusable bag for retrieval of a mass, such as an excised organ, through a laparoscopic sleeve and also allows for morsellizing the severed organ.

U.S. Pat. No. 6,270,505 to Yoshida et al. discloses an inflatable endo-bag for organ removal, ports for surgical instruments and a closing string.

All of the prior art references discussed above are drawn to encapsulation of an organ after it has been surgically severed from its attachments to the body. In particular, all of these prior art references address the problem of transport of an organ outside the abdominal cavity after it has been laparoscopically excised inside the abdominal cavity. Accordingly, these prior art references do not address the special problem of separating a mass, such as a cyst, from a live, intact organ such as an ovary without rupture of the cyst and spillage of its contents.

U.S. Pat. No. 5,279,539 to Bohan et al. discloses a bioabsorbable surgical pouch which can be placed over an ovary after a surgical procedure to prevent surgical adhesions. However, Bohan et al. do not disclose the use of the pouch during a surgical procedure such as performing an ovarian cystectomy.

There are typically two steps involved in the removal of an ovarian cyst by laparoscopy. The first step is the separation of the cyst from the ovary. The second step is the transport of the cyst outside of the abdominal cavity once it has been separated from the ovary. There are problems associated with each step. The problem in the first step is how to separate the cyst from the ovary without rupturing the cyst and spilling its contents. The problem with the second step is how to transport a large cyst outside the abdominal cavity through a small opening in the abdomen. All of the prior art discussed above are involved only with the problem associated with the second step, that is, transport of a mass or organ. outside the abdomen after it has been excised.

### SUMMARY OF THE INVENTION

Preferred embodiments of the present invention address the first problem, specifically, separation of a mass such as a cyst from an ovary without spilling its contents into the abdominal cavity. It will be appreciated, however, that the methods and apparatuses discussed herein are not limited to ovarian cystectomies, and therefore, may have applicability to any procedure wherein it is desired to remove biological or other material from an organ, or to remove an organ itself, in a contained environment.

Several unique features are disclosed by the preferred embodiments discussed below. First, in one embodiment, an intact organ is encapsulated, preferably with a non-permeable bag-type or other expandable structure, while it is still attached to the body. As used herein, an intact organ is defined as an organ that is still attached to the body as found in its natural state. The intact organ is preferably a live intact organ. However, intact organs which contain dead tissue are also encompassed within the present invention. The encapsulation of an intact organ is in contrast to the prior art references discussed above, wherein encapsulation can only occur after the organ or mass has been completely separated from its attachment to the body. Thus, in the prior art discussed above, the organ or mass is first completely excised and then encapsulated. In preferred embodiments of the present invention, the organ or mass is first encapsulated and then the process of excision is performed. These embodiments advantageously allow a mass, such as an ovarian cyst, for example, to be separated from the ovary without spilling its contents into the abdominal cavity.

In one embodiment, the disclosed bag or other expandable structure surrounding the intact organ is provided with multiple openings for insertion of one or more instruments. These instruments may be inserted simultaneously to perform operations on an intact organ such as removal of a cyst from an ovary, or other desired procedures. In one embodiment, openings are provided with a one-way valve. In other embodiments, the expandable structure expands to form a bowl or cup and is placed underneath the intact organ. In these alternative embodiments, the expandable structure does not completely enclose the intact organ.

In one embodiment, the bag is a balloon-like structure designed to prevent spillage of the cyst content into the peritoneal cavity and thus make laparoscopic ovarian cystectomy safe and easy. The balloon-like structure preferably comprises an inflatable, water-tight, fire-retardant bag with an opening on a distal side thereof and a gas nozzle on a proximal side thereof. In a preferred embodiment, the bag is funnel-shaped and has a large noose-like opening at the distal end. In one embodiment, the proximal end is long and tapered. In a preferred embodiment, the distal end has a relatively large opening, at least large enough to encapsulate an ovary or other organ.

The bag in one embodiment used for laparoscopic ovarian cystectomy is introduced into the peritoneal cavity through a laparoscopic port, the proximal end with the gas nozzle being retained on the outside of the body for gas infusion. Once inside the peritoneal cavity, the large distal opening of the bag is then placed over the ovary and fastened. In a preferred embodiment, a noose is tightened down over the ovarian pedicle and locked in using a fastening device such as a trumpet valve. As used herein, the term “pedicle” is used in its ordinary sense and refers to any stem-like structure that extends from an organ. The term “ovarian pedicle” is used in its ordinary sense and should be interpreted to include a combination of the infundibulo-pelvic ligament, the utero-ovarian ligament and/or the meso-ovarian ligament. In the presence of an ovarian cyst, these tissues can become elongated to form a slender stem-like structure that is herein referred to as the ovarian pedicle. Tightening the noose around this ovarian pedicle allows the bag to completely envelop the ovary and its cyst and form an air-tight seal. In a preferred embodiment, gas is then infused through the gas nozzle to inflate the bag.

In a preferred embodiment, a laparoscopic camera and other instruments can now be passed into the inflated bag through one-way valves to operate on the cyst. Any leakage of cyst content during the surgery is now contained within the bag and therefore does not come into contact with the peritoneal cavity and its organs. In a preferred embodiment, the cyst can be deflated without any concern for contamination and the cystectomy performed much easier and faster on a collapsed cyst. An additional advantage of the bag is that it also prevents accidental and harmful contact between the laparoscopic instruments and other organs such as the bowel or bladder.

After the cystectomy is completed, any fluid leakage into the bag is removed by laparoscopic suction and the bag thoroughly irrigated. The noose is then loosened and the ovary removed from the bag. The noose is closed again and the bag, containing the specimen, is removed from the peritoneal cavity through a laparoscopic port.

The preferred methods and apparatuses described above and in further detail below allow for a safe, rapid and easy laparoscopic ovarian cystectomy because it circumvents the most difficult and time-consuming aspect of the surgery, i.e., the prevention of cyst spillage into the peritoneal cavity.

For purposes of summarizing the invention and the advantages achieved over the prior art, certain objects and advantages of the invention have been described above. Of course, it is to be understood that not necessarily all such objects or advantages may be achieved in accordance with any particular embodiment of the invention. Thus, for example, those skilled in the art may recognize that the invention may be embodied or carried out in a different manner that achieves or optimizes one advantage or group of advantages as taught herein without necessarily achieving other objects or advantages as may be taught or suggested herein.

Accordingly, in one embodiment, a surgical device is disclosed for enclosing an intact organ to perform a surgical procedure thereon. The device includes a non-permeable material adapted to be closed over an intact organ while substantially enclosing the intact organ and at least one port in the non-permeable material adapted to allow a surgical instrument to pass therethrough to operate on the intact organ. In a preferred embodiment, the non-permeable material is an inflatable bag and preferably includes a nozzle at one end for inflating the bag while the material is closed over the intact organ.

In a preferred embodiment, the intact organ is an ovary.

In one embodiment, a surgical device for enclosing an intact organ to perform a surgical procedure thereon is disclosed which includes an inflatable bag comprising flexible material and having an opening at a distal end thereof sufficiently large to receive the intact organ and a nozzle at a proximal end thereof adapted to inflate the bag and at least one port which includes a one-way valve to allow a surgical instrument to be introduced into the bag to perform a surgical operation on the intact organ contained within the bag without leakage of gas. In a preferred embodiment, the intact organ is an ovary.

In a preferred embodiment the surgical device includes an insertion housing for enclosing the bag. In a preferred embodiment, the surgical device also includes a plurality of ports to allow surgical instruments to be introduced into the bag.

In another preferred embodiment, the invention includes a surgical device for enclosing an intact organ to perform a surgical procedure thereon, the device including:

an expandable structure having a collapsed configuration and an expanded configuration;

a membrane attached to the expandable structure; and

at least one port in the membrane adapted to allow a surgical instrument to pass therethrough to operate on the intact organ.

In a preferred embodiment, the membrane of the surgical device which is attached to the expandable structure is substantially funnel-shaped when the expandable structure is in its expanded configuration.

Preferably, the intact organ is a live intact organ. More preferably, the intact organ is an ovary. In a preferred embodiment, the membrane is adapted to be closed over a pedicle of the intact organ.

In a preferred embodiment, the expandable structure is self-expanding. Preferably, the expandable structure includes a plurality of self-expanding struts.

Preferred embodiments of the invention include a surgical device for enclosing an intact organ to perform a surgical procedure thereon, which includes:

an expandable structure including a plurality of struts;

a membrane attached to the struts, having an opening at a distal end thereof sufficiently large to receive the intact organ and an attachment point at a proximal end thereof adapted to manipulate and retrieve the expandable structure; and

at least one port to allow a surgical instrument to be introduced into the expandable structure to perform a surgical operation on the intact organ contained within the expandable structure.

In a preferred embodiment, the struts are self-expanding. In some preferred embodiments, the struts include spring-loaded metal prongs. In a preferred embodiment, the attachment point is a flexible loop.

Preferably, the intact organ is a live intact organ. More preferably, the intact organ is an ovary.

In a preferred embodiment, the surgical device includes a drawstring near the opening to close the opening. In a preferred embodiment, the opening at the distal end is a noose-like opening. Preferably, the surgical device includes an insertion housing for enclosing the expandable structure. Preferably, the surgical device includes a plurality of ports to allow surgical instruments to be introduced into the expandable structure.

In another embodiment a method of performing a cystectomy is disclosed which includes the steps of: (1) introducing an inflatable bag into an abdominal cavity of a patient, wherein a distal end of the inflatable bag has an opening sufficiently large to encapsulate an ovary which is attached to the body; (2) positioning the inflatable bag over the ovary to encapsulate the ovary; (3) closing the opening of the inflatable bag over an ovarian pedicle; (4) inflating the bag; and (5) introducing one or more instruments into the bag to separate a cyst from the encapsulated ovary.

In a preferred embodiment, the method includes inserting the bag into the abdominal cavity within an insertion housing.

In another embodiment, a method of performing an operation on an intact organ, is disclosed which includes the steps of: (1) introducing a non-permeable structure into a body space near to the intact organ; (2) enclosing at least a portion of the intact organ with the non-permeable structure, the non-permeable structure defining a working space between the intact organ and the non-permeable structure; and (3) introducing one or more instruments through the non-permeable structure to perform the operation on the intact organ within the working space. In some embodiments where the intact organ is only partially enveloped, the instruments do not have to pass through the non-permeable structure in order to access the intact organ.

Other preferred embodiments of the invention include a method of performing a cystectomy, which includes the steps of:

introducing an expandable structure having a frame attached to a membrane capable of having a collapsed configuration and an expanded configuration into an abdominal cavity of a patient;

expanding the frame attached to the membrane to the expanded configuration to provide an opening at the distal end of the frame sufficiently large to encapsulate an ovary which is attached to the body;

positioning the frame over the ovary to encapsulate the ovary;

closing the opening of the expandable structure over an ovarian pedicle; and

introducing one or more instruments into the expandable structure to separate a cyst from the encapsulated ovary.

In a preferred embodiment, the method includes removing the expandable structure from the ovarian pedicle and releasing the ovary from the expandable structure. More preferably, the method includes re-closing the opening of the expandable structure with the ovary outside the expandable structure and with the removed cyst inside the expandable structure. More preferably, the method includes transporting the separated cyst outside of the abdominal cavity within the expandable structure.

In a preferred embodiment, the method includes introducing the expandable structure into the abdominal cavity comprises inserting the expandable structure into the abdominal cavity within an insertion housing.

In a preferred embodiment, the expandable structure is opened sufficiently to allow for the performance of the cystectomy inside the expandable structure.

In a preferred embodiment, closing the opening of the expandable structure over the pedicle is performed by pulling a drawstring closed over the ovarian pedicle.

In a preferred embodiment, introducing one or more instruments into the expandable structure is performed by inserting said instruments through ports in a surface of the membrane.

In other embodiments, the surgical device for performing a surgical procedure, includes an expandable structure including a plurality of struts, and a membrane attached to the struts, having an opening at a distal end thereof and an attachment point at a proximal end thereof adapted to manipulate and retrieve the expandable structure, wherein the expandable structure in an expanded configuration forms a bowl to collect leakage from the surgical procedure.

In some embodiments, the struts include spring-loaded metal prongs. In preferred embodiments, the struts are self-expanding. Preferably, the attachment point includes a flexible loop. Preferably, the surgical device also includes an insertion housing for enclosing the expandable structure.

In some embodiments, a method of performing a cystectomy is disclosed which includes the steps of:

introducing an expandable structure capable of having a collapsed configuration and an expanded configuration into an abdominal cavity of a patient;

expanding the frame attached to the membrane to the expanded configuration to provide a bowl;

positioning the bowl underneath an ovary to collect leakage from the cystectomy; separating a cyst from the ovary; and

suctioning the leakage from the bowl.

Preferably, the step of introducing the expandable structure into the abdominal cavity includes inserting the expandable structure into the abdominal cavity within an insertion housing. Preferably, the expandable structure includes a frame attached to a membrane.

Further aspects, features and advantages of this invention will become apparent from the detailed description of the preferred embodiments which follow.

### BRIEF DESCRIPTION OF THE DRAWINGS

These and other feature of this invention will now be described with reference to the drawings of preferred embodiments which are intended to illustrate and not to limit the invention.

FIG. 1A is a perspective view of a bag according to one embodiment of the present invention.

FIG. 1B is a perspective view of a bag having a trumpet valve according to another embodiment of the present invention.

FIG. 2 is a side view of an insertion housing for inserting the bag of FIG. 1A or 1B, with the bag shown inside the insertion housing in phantom.

FIG. 3 is a schematic view of the bag of FIG. 1A after insertion into the abdomen via the suprapubic port under visualization by a laparoscopic camera.

FIG. 4 is a schematic view of the bag of FIG. 3 after placement around an ovary and cyst and after a noose on the bag has been tightened down.

FIG. 5 is a schematic view of instruments inserted into the inflated bag through one-way valves under guidance by the laparoscopic camera.

FIG. 6A is a schematic view of the inflated bag within the abdominal cavity, showing the laparoscopic instruments and camera inside the bag, ready to operate on the cyst.

FIG. 6B is a schematic view showing surgery inside the bag, in this case, puncturing and decompressing the cyst.

FIG. 7 is a schematic view of the interior of the bag after the cyst has been separated from the ovary and the spilled cyst content is being removed from the bag via laparoscopic suction.

FIG. 8 is a schematic view of the bag after deflation, wherein the instruments and the ovary are removed from the bag, the noose is tightened down to close the bag and the gas nozzle is inserted into the abdomen.

FIG. 9 is a schematic view of the deflated bag containing the cyst as it is removed from the abdomen through a laparoscopic port.

FIG. 10 is a schematic view of a self-expanding endoscopic bag for spill-proof laparoscopic ovarian cystectomy.

FIG. 11 is a schematic view of the self-expanding endoscopic bag for spill-proof laparoscopic ovarian cystectomy of FIG. 10 within an insertion housing.

FIG. 12 is a schematic view of the self-expanding endoscopic bag for spill-proof laparoscopic ovarian cystectomy of FIG. 10 placed around an ovary with the noose tightened around the ovarian pedicle.

FIG. 13 is a schematic view of the self-expanding endoscopic bag for spill-proof laparoscopic ovarian cystectomy of FIG. 10 after the cyst is removed and fluid has been suctioned out.

FIG. 14 is a schematic view of the self-expanding endoscopic bag for spill-proof laparoscopic ovarian cystectomy of FIG. 10 as it is pulled into the trochar sleeve.

FIG. 15 is a schematic view of an expandable endoscopic bag for spill-proof laparoscopic ovarian cystectomy.

FIG. 16 is a schematic view of the expandable endoscopic bag of FIG. 15 during an ovarian cystectomy.

### DETAILED DESCRIPTION OF THE PREFERRED EMBODIMENTS

While the described embodiments represent preferred embodiments of the present invention, it is to be understood that modifications will occur to those skilled in the art without departing from the spirit of the invention. The scope of the invention is therefore to be determined solely by the appended claims.

Briefly stated, preferred embodiments relate to a bag or expandable structure used to at least partially encapsulate an ovary for performing a cystectomy, and methods for delivering the bag or expandable structure and for performing the cystectomy. The bag or expandable structure may encapsulate the ovary or may provide a bowl-like structure underneath the ovary to collect leakage and other materials that result from the operation. It will be appreciated, however, that the methods and apparatuses taught herein are not limited to ovarian cystectomies, and thus, other procedures may benefit from the embodiments described herein.

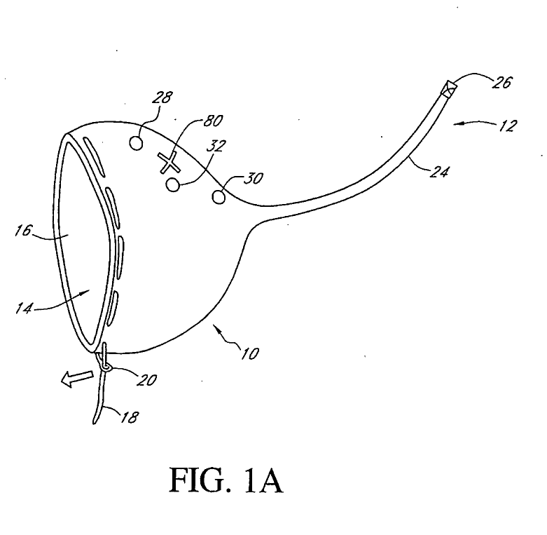

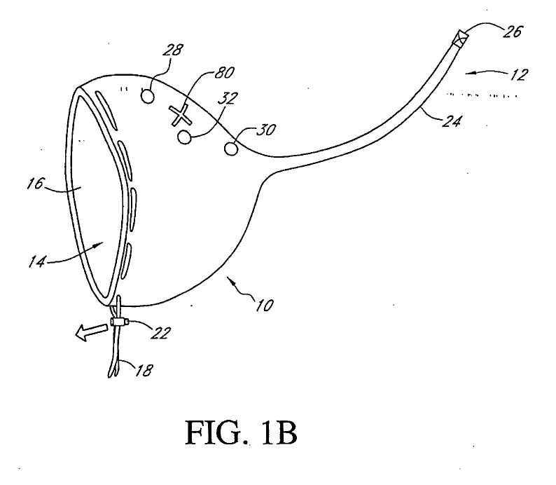

FIG. 1A shows one embodiment of a bag 10 to be used to perform the cystectomy. The bag as illustrated is generally funnel-shaped and has a proximal end 12 and a distal end 14. At the distal end 14, an opening 16 is provided which is sized and configured to fit over an organ such as an ovary. As illustrated, the opening 16 can be circular, or may have any other shape as desired that is adapted to form a substantially air tight seal with a portion of an organ such as an ovarian pedicle. In one embodiment, a drawstring 18 is provided near the edge of the opening 16, and is adapted to be pulled taut over a tissue pedicle to seal and lock the bag. The drawstring 18 may be made of any stretchable, rubber-band-like material or non-stretchable material including but not limited to an elastic band, rubber band, suture material, etc., or any other desired material.

In the embodiment illustrated in FIG. 1A, to close the bag over a pedicle, the drawstring 18 is pulled through a noose 20 to seal and lock the bag. Other embodiments may also be used to seal and lock the bag, such as a noose over a button, a velcro fastening or a pre-tied suture. FIG. 1B illustrates one embodiment wherein the drawstring 18 can be closed using a trumpet valve 22. It will be appreciated that other means may be used to seal the bag 10. In one embodiment, closure of the opening 16 of the bag 10 over a tissue pedicle may be further reinforced with a separate rubber band placed around the outside of the bag near the opening 16.

The bag itself may be made of any flexible material that is capable of being blown up like a balloon including, but not limited to, plastic material, latex, or rubber. The balloon material may be compliant in order to fill the desired space within the patient, or may be non-compliant to inflate to a predetermined size. It will be appreciated, however, that the invention is not to be limited to inflatable structures. Any non-permeable structure capable of isolating an organ to be treated from surrounding organs or bodily space will be suitable in performing the cystectomy or other procedure. For example, expandable structures (both self-expanding and non-self-expanding) may be used.

At the proximal end 12 of the bag 10, away from the opening 16, the bag preferably tapers into a long tube 24. At or near the very proximal end of the tube 24, a gas nozzle 26 may be provided to allow for inflation of the bag. The tube 24 and the gas nozzle 26 are preferably adapted to extend outside of the body and allows for infusion of gas to blow up the bag 10 when the opening is sealed over a tissue pedicle. The gas nozzle 26 preferably includes a one-way valve to allow gas or other fluid to be delivered into the bag and maintain inflation of the bag.

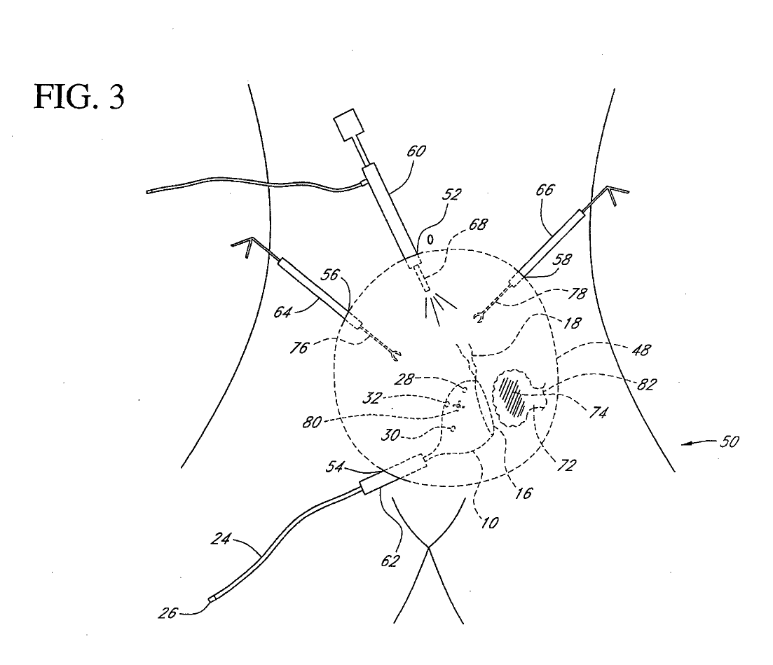

As illustrated in FIG. 1A, the bag may also have additional openings or ports 28, 30, and 32 (see also FIGS. 3-9) to allow for insertion of surgical instruments, as described below. These ports 28, 30, and 32 may be placed at desired locations on the outside of the bag to allow for the insertion of appropriate surgical instruments at desired locations. Preferably, each of these ports includes a one-way valve, as described below, to prevent gas inside the bag from escaping when an instrument is inserted therethrough.

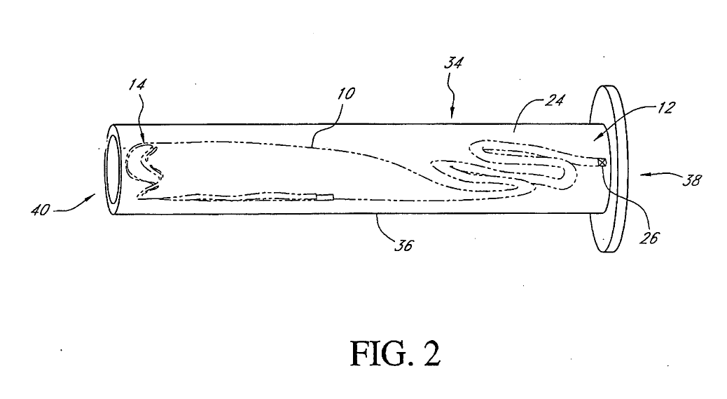

FIG. 2 illustrates an insertion housing 34 used in one embodiment to insert the bag 10 into a patient. The insertion housing preferably includes a tubular body 36 having a proximal end 38 corresponding to the proximal end 12 of the bag 10, and a distal end 40 corresponding to the distal end 14 of the bag 10. The insertion housing may be made of any relatively hard, biologically non-reactive material including but not limited to plastic materials. FIG. 2 illustrates the bag 10 inside the insertion housing 34 before insertion into the body in a compressed configuration. The distal end 40 of the housing is preferably open and the proximal end 38 of the housing is preferably closed prior to delivery of the bag 10. Further details regarding delivery of the bag into a patient using the insertion housing 34 are described below.

FIG. 3 illustrates one embodiment for inserting the bag 10 into the peritoneal or abdominal cavity 48 of a patient 50. As illustrated, a plurality of incisions ranging from 5-10 mm 52, 54, 56, 58 are made in the patient 50. Then, using standard laparoscopic techniques, trocars and trocar sleeves are inserted into the abdomen. After the trocars are removed, the trocar sleeves 60, 62, 64 and 66, as described further below, remain extending from the outside of the patient. The trocar sleeves 60, 62, 64 and 66 each preferably includes a one-way valve such that when instruments are passed through the trocar sleeves into the abdomen of the patient, gas from inside the abdomen, as described below, does not escape. The trocar sleeves described in the embodiments herein preferably have a size of 5 mm or 10 mm. However, it will be appreciated that any size trocar sleeve may be used that is appropriate to carry out the surgical procedure. Different ports and adapters may also be used as needed.

In the embodiment illustrated in FIG. 3, the laparoscope 68 is preferably inserted into the abdomen through a trocar sleeve 60 near the navel. Gas, preferably carbon dioxide, is introduced into the abdominal cavity via one of the trocar sleeves, such as sleeve 60. The intra-abdominal pressure is preferably maintained at a constant level using well-known methods and apparatus.

The bag 10 is preferably introduced into the abdominal cavity 48 through a second 5 or 10 mm trocar sleeve 62 via a suprapubic incision 54 or any other location as desired by the surgeon. In one embodiment, the insertion housing holding the bag, described above, is inserted through the trocar sleeve 62 until its distal end 14 (corresponding to the opening of the bag 16) enters the abdominal cavity as shown in FIG. 3. In a preferred embodiment, the outer diameter of the housing is a perfect fit with the inner diameter of the trocar sleeve 62 such that when the housing is passed through the trocar sleeve, CO₂ gas from within the abdomen is prevented from escaping due to the housing 34 being sealed at its proximal end 38.

After the housing is delivered through the trocar sleeve 62 into the abdomen, the bag is then grasped from the distal end of the insertion housing with a laparoscopic grasper 78 while the insertion housing 34 is pulled out, leaving the gas nozzle 26 on the outside of the body. Preferably, the gas nozzle 26 will be of such size and material such that it will form a tight seal with the one-way valve of the trocar sleeve 62. A 10-5 mm seal reducer may be used if necessary to achieve a tight seal. It will be appreciated that other methods and apparatuses may be used to insert the bag 10 into the abdomen 48 of a patient 50.

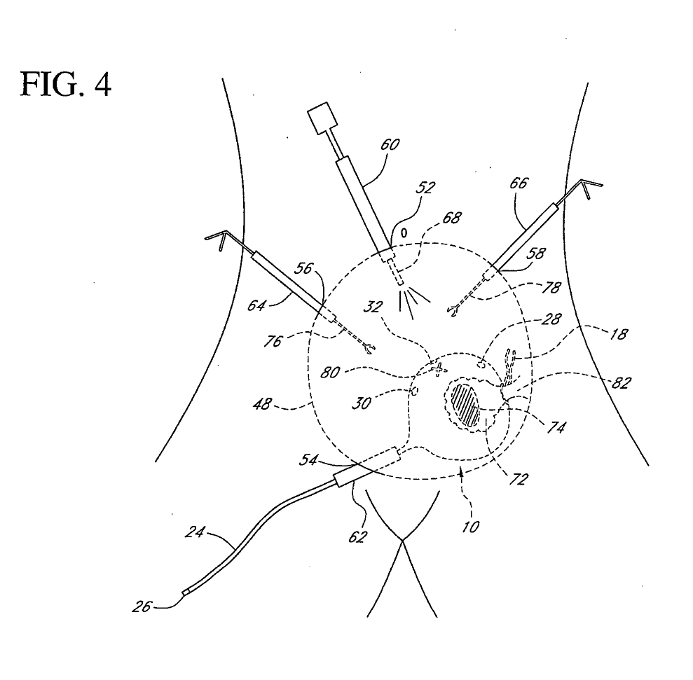

As shown in FIG. 4, in performing an ovarian cystectomy, once inside the abdominal cavity the bag 10 is preferably maneuvered such that the opening 16 of the bag 10 is placed over the ovary 72 having a cyst 74 and the ovarian pedicle 82. Positioning of the bag is facilitated by the use of laparoscopic instruments 76 and 78 that can be inserted into the abdomen. In one embodiment, ports 28, 30, and 32 containing one-way valves on the bag 10 are generally lined up, respectively, with the trocar sleeves 66, 64, and 60, extending into the patient's abdomen, so that the laparoscope 68 and other laparoscopic instruments 76, 78 passing through these sleeves can be directly inserted into these ports 28, 30, and 32. The bag 10 may also have one or more external markings 80 to help guide the alignment of the ports on the bag 28, 30, 32 with the trocar sleeves 66, 64, and 60. As shown in FIG. 4, a mark 80 on the bag 10 may be used to align the port 32 with the trocar sleeve 60 and the laparoscope 68.

Once the opening 16 is positioned over the ovary 72 and the ovarian pedicle 82 and the bag is properly aligned, the opening is closed over the ovarian pedicle as shown in FIG. 4. Closure can occur using a noose 20 or other methods as described with respect to FIGS. 1A and 1B above. Laparoscopic instruments such as grasping forceps 76, 78 may be used to assist in closing the bag 10.

Gas, preferably carbon dioxide, is then infused into the bag 10 via the gas nozzle 26 to inflate the bag. The bag 10 may be inflated as required to provide a space to perform the surgery and in one embodiment, may be inflated up against the abdominal wall 48.

With the bag inflated, the ports 28, 30 and 32 on the bag are preferably aligned with the incisions 58, 56 and 52 and the trocar sleeves 66, 64 and 60, respectively. If the bag is not properly aligned upon initial inflation, the physician can use grasping instruments such as grasping forceps 76, 78 which are inserted into the abdominal cavity 48 but have not yet been inserted into the bag to finalize the alignment of the bag.

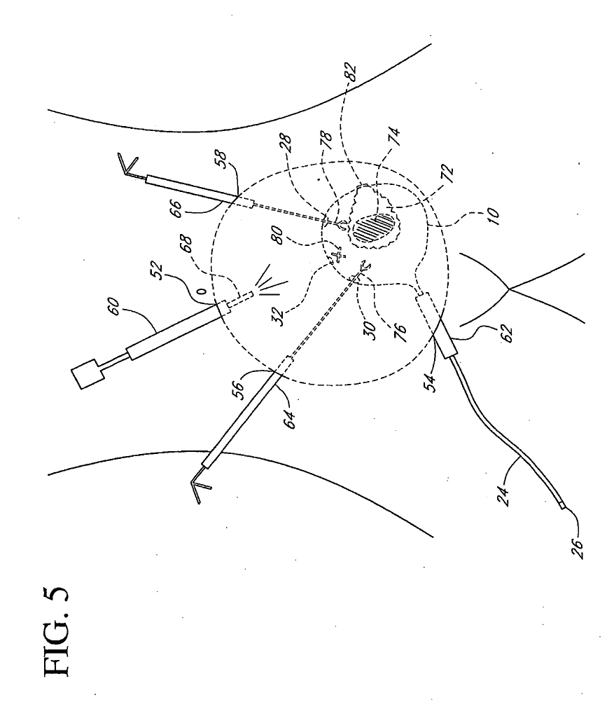

FIG. 5 illustrates the bag after it has been inflated in the abdominal cavity 48 with instruments inserted into the bag 10. The laparoscope remains outside of the bag 10, but inside the abdominal space to allow the operator to visualize where the instruments will be inserted through the bag. Instruments, including but not limited to grasping forceps, blunt probes, needle electrodes or laparoscopic scissors, etc. may be inserted into the ports 28 and 30 on the bag 10. Each of these ports 28 and 30 preferably includes a one-way valve to allow for insertions of the instruments without leakage of gas.

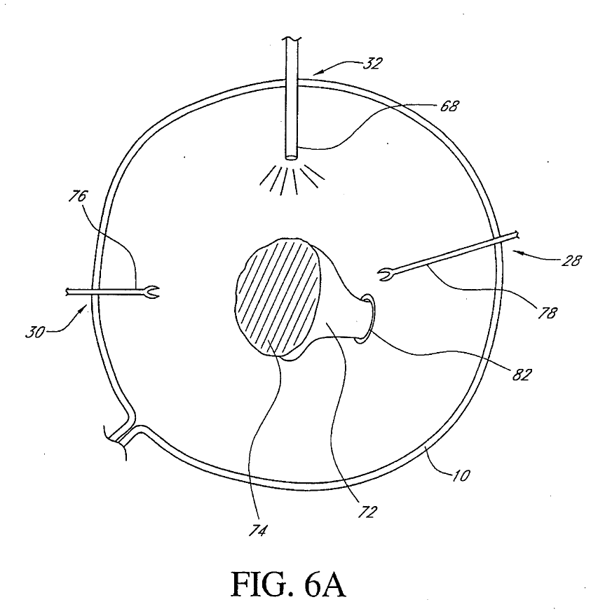

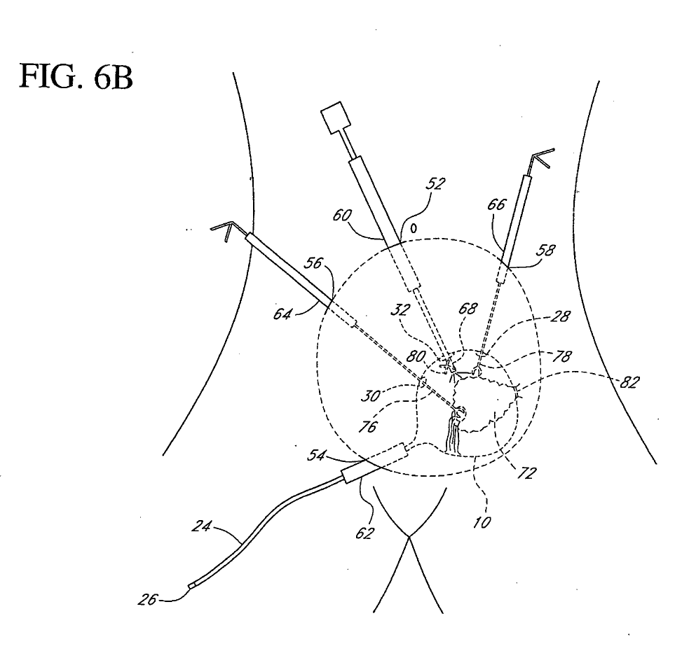

As shown in FIGS. 6A and 6B, after the instruments 76 and 78 are inserted into the bag 10, the laparoscope 68 is then inserted through a port 32 in the bag 10 to provide visualization for the procedure inside the bag 10. The cystectomy or other procedure is then performed under direct visualization by methods well known to those skilled in the art. More instruments may be inserted into the bag to help facilitate the procedure if so desired by the surgeon.

In one preferred embodiment, after the laparoscope 68 and instruments 76 and 78 are inserted into the bag 10, the cyst 74 can safely be punctured and deflated before excision using one of the instruments (FIG. 6B). This is in contrast to procedures performed in the past, where physicians would use great care not to puncture the cyst to avoid leakage into the peritoneal cavity. In this embodiment of the invention, any leakage of fluid is advantageously contained within the bag. This allows the cystectomy to be safely and easily performed by the laparoscopic method, regardless of the nature of the cyst. In fact, no matter what type of cyst is present, with the preferred embodiments of the invention described above, there is no leakage of fluid into the peritoneal cavity. Consequently, even an inexperienced surgeon can perform a laparoscopic ovarian cystectomy using the bag described above because any fluid leakage from a malignant, dermoid or mucinous cyst is retained safely within the bag. Another advantage with a preferred embodiment of the invention is that because of the ability to deflate the cyst safely, even a very large cyst which formerly would have required a laparotomy even by an experienced surgeon, can now be easily removed by laparoscopy.

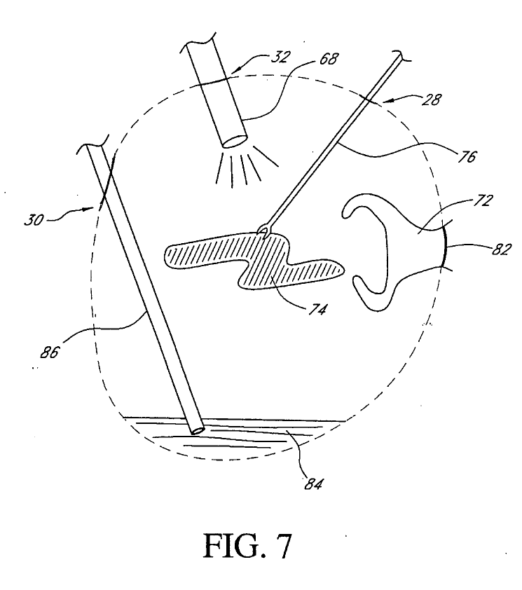

Once the cyst is punctured and the fluid has leaked out, the cyst 74 often becomes easier to remove from the ovary 72. As shown in FIG. 7, the punctured cyst 74 can be removed and separated from the ovary using known techniques. Alternatively, should the surgeon prefer, the cystectomy can proceed without first deflating the cyst. However any accidental cyst leakage during the procedure would still be contained within the bag 10. In a preferred embodiment, any spillage 84 caused by the procedure is removed with a laparoscopic suction-irrigator 86 and the bag is thoroughly cleansed with irrigation. In one embodiment, a suction-irrigator 86 is inserted through one of the ports 30 in the bag 10 to irrigate and remove any loose substances or fluid. After irrigation and suction of the fluid in the bag, the instruments 76, 86 and laparoscope 68 may be removed through the ports 28, 30, and 32.

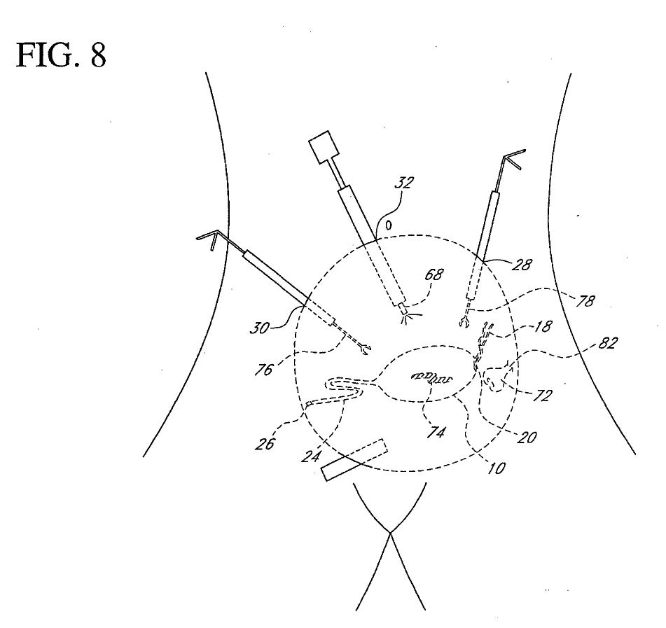

As shown in FIG. 8, after irrigation, suction, and instrument removal, the bag is deflated while the noose over the ovarian pedicle 82 is loosened to allow removal of the ovary 72 from the bag 10. The noose 20 can then be tightened again using the surgical instruments 76, 78 which are now outside the bag but still within the abdominal space to seal off the bag 10 with the excised cyst 74 inside as shown in FIG. 8. In a preferred embodiment, as described above, the cyst 74 has been punctured and deflated. Consequently, it is now much smaller and the bag 10 containing it may be easily removed through one of the trocar sleeves. However, in some embodiments where the tissue mass is still large, it may be desirable to reduce the size of the tissue prior to deflating the bag by grinding the tissue with an instrument designed for that purpose such as a morsellizer (see U.S. Pat. No. 6,059,793 to Pagedas which is incorporated herein by reference).

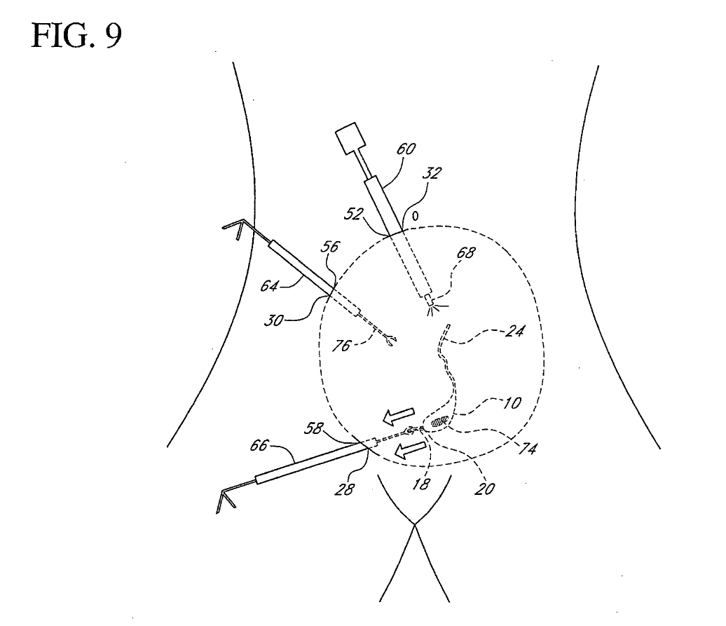

The bag is desirably removed through one of the trocar sleeves inserted through one of the laparoscopic incisions. In one embodiment, the tube 24 and the gas nozzle 26 of the bag 10 are pushed into the abdomen as shown in FIG. 8. FIG. 9 shows removal of the deflated bag 10 containing the cyst 74 through the trocar sleeve 66. It will be appreciated that the bag can be removed through any of the trocar sleeves. In a preferred embodiment, the bag is grabbed by the tightened noose 20 and the bag 10 with cyst 74 can be easily removed without contamination of the peritoneal cavity.

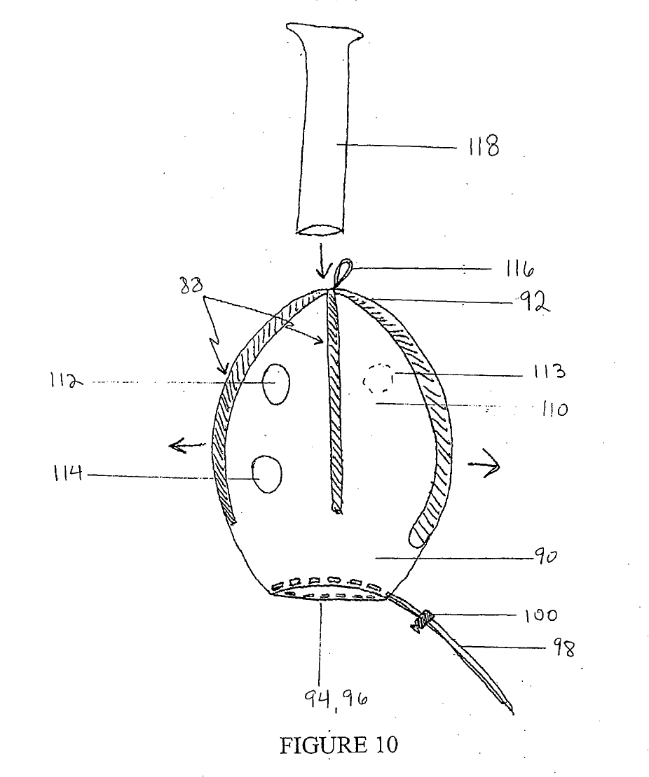

FIG. 10 shows an alternate embodiment of an expandable structure used for spill-proof laparoscopic ovarian cystectomy. The expandable structure 110 includes a plurality of struts 88 defining a frame with a membrane 90, more preferably made of a non-permeable material, attached to the struts 88. The plurality of struts 88 have a common proximal point 92 and extend towards the distal end 94 of the structure but are not attached at the distal end 94. The struts 88 may be made from metal or plastic material including but not limited to one or more elements of high strength material such as stainless steel or MP35N, or may preferably be made from shape memory or pseudoelastic alloys such as NiTi, or any of a variety of known structure biodegradable materials (e.g. polyglycolic acid, polylactic acid, poly-L-lactic acid and derivatives or copolymers such as PLGA).

Preferably, the struts 88 are self-expanding between a collapsed configuration and an open or expanded configuration, although they may also be mechanically deployed. In the expanded configuration, the struts 88 expand radially outward to define a generally spherical or funnel-shaped volume. The struts 88 may be integrally formed, or may be separately attached to a hub at proximal point 92. The membrane 90 may be attached to the struts 88 by a suitable method such as gluing, welding, sewing or other suitable method, and may expand to an open configuration to define an interior volume through movement of the struts. The membrane 90 may be elastic or inelastic, and may comprise a single membrane attached to one side of the struts or a laminate structure applied to both sides of the struts. For example, the membrane 90 may be made of any material that is suitable for expansion with the struts, including, but not limited to, fabric, plastic material, latex, or rubber.

The struts 88 open radially outward to expand the membrane 90. The expandable structure as shown has a proximal 92 and a distal 94 end. At the distal end 94, an opening 96 is provided which is sized and configured to fit over an organ such as an ovary. As illustrated, the opening 96 can be circular, polygonal or may have any other shape as desired that is adapted to enclose an organ such as an ovarian pedicle.

In one embodiment, a drawstring 98 is provided near the edge of the opening 96 and is adapted to be pulled taut over a tissue pedicle to seal and lock the bag. The drawstring 98 may be made out of any stretchable, rubberband-like material or a non-stretchable material including but not limited to an elastic band, rubber band, suture material, etc., or any other desired material.

As illustrated in FIG. 10, the drawstring 98 is closed using a trumpet valve 100. It will be appreciated that other means may be used to close the expandable structure 110 as discussed above.

As illustrated in FIG. 10, the expandable structure may also have additional openings or ports 112, 113 and 114 to allow for insertion of surgical instruments as described above. These ports 112, 113 and 114 may be placed at desired locations on the outside of the expandable structure 110 to allow for the insertion of appropriate surgical instruments at desired locations. More particularly, these ports may be provided on the membrane 90 in a similar manner to that of the inflatable bag as described above.

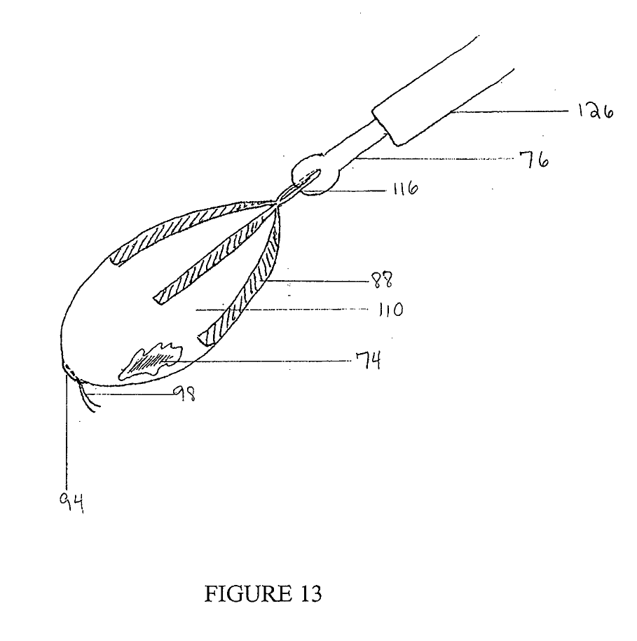

The expandable structure 110 may be converted from the expanded configuration to the collapsed configuration and withdrawn from the body cavity via the loop 116 at the proximal end 92 of the expandable structure. This loop may be separately attached to the struts, or may be integrally formed with the struts. The loop may be used to attach the expandable structure to a laporoscopic device (shown in FIG. 13), used to deliver and retrieve the expandable structure from the body.

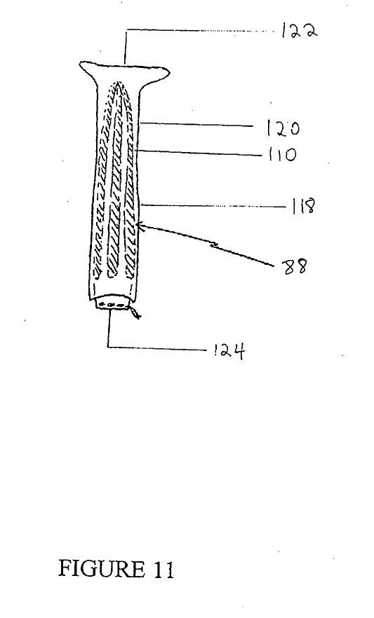

The expanded configuration is shown in FIG. 10 illustrates an insertion housing 118 used in one embodiment to insert the expandable structure 110 into a patient. As illustrated in more detail in FIG. 11, the insertion housing is similar to the insertion housing described above and includes a tubular body 120 having a proximal end 122 corresponding to the proximal end 92 of the expandable structure 110 and a distal end 124 corresponding to the distal end 94 of the expandable structure. The insertion housing may be made of any relatively hard, biologically non-reactive material as discussed above, including but not limited to plastic materials.

In a collapsed configuration, the expandable structure 110 is adapted to fit inside an insertion housing 118. FIG. 11 illustrates the expandable structure 110 inside the insertion housing 118 before insertion into the body in the collapsed configuration. In one embodiment the struts 88 are squeezed together like a spring. While this embodiment is self-expanding, non-self-expanding expandable structures are also within the scope of the invention. Similar to the inflatable bag embodiment described above, the expandable structure 110 can be removed from the housing by using laporoscopic instruments to pull the expandable structure 110 out of the housing. Upon removal from the housing, the expandable structure 110 in the embodiment illustrated self-expands to its open configuration.

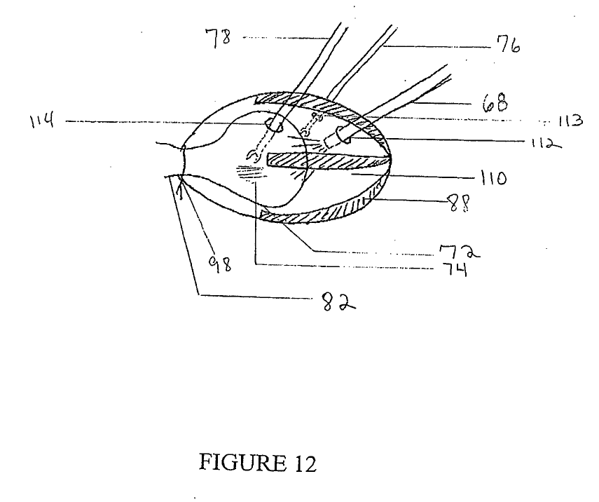

FIG. 12 illustrates the expandable structure 110 placed around an ovary 72 having a cyst 74 with the drawstring 98 tightened around the ovarian pedicle 82. Positioning of the bag is facilitated by the laparoscope 68 and the laporoscopic instruments 76 and 78 that can be inserted into the abdomen. In one embodiment, the ports 112, 113, and 114 on the membrane 90 attached to the struts 88 of the expandable structure 110 are generally lined up with the trocar sleeves 66, 64, and 60 (not shown in FIG. 12) discussed above. As discussed with respect to other embodiments above, one or more external markings help in alignment of the ports with the trocar sleeves. Further details regarding procedures and techniques that can be used are described with respect to the inflatable bag embodiment above.

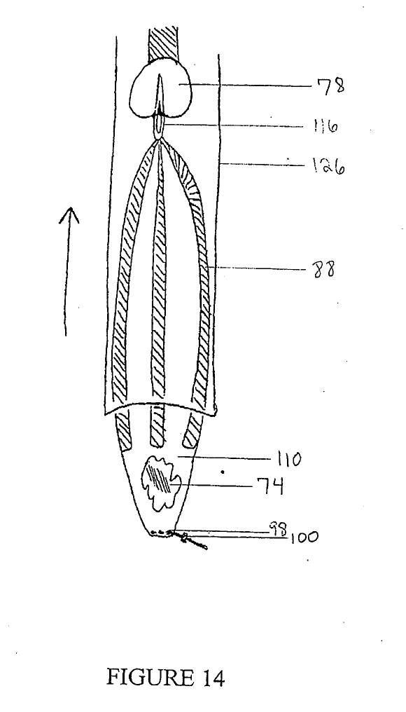

FIG. 13 illustrates the expandable structure 110 after the cyst 74 is removed and all of the fluid has been suctioned out, as described in the embodiment illustrated above. The ovary has been removed from the expandable structure 110 and the drawstring 98 tightened to close the opening 96 at the distal end 94 of the expandable structure 110. The expandable structure 110 is then pulled out of a body cavity through a trocar sleeve 126, using a laporoscopic grasper 76 engaging the loop 116. In one embodiment, as the expandable structure is pulled out of the body cavity into the trocar sleeve, the struts 88 pull together and collapse within the trocar sleeve to the collapsed configuration. FIG. 14 illustrates as the expandable structure 110 enclosing the cyst 74 is pulled into the trocar sleeve 126, the struts 88 are squeezed together to allow the expandable structure 110 to be brought to the outside of the body cavity through the trocar sleeve 126.

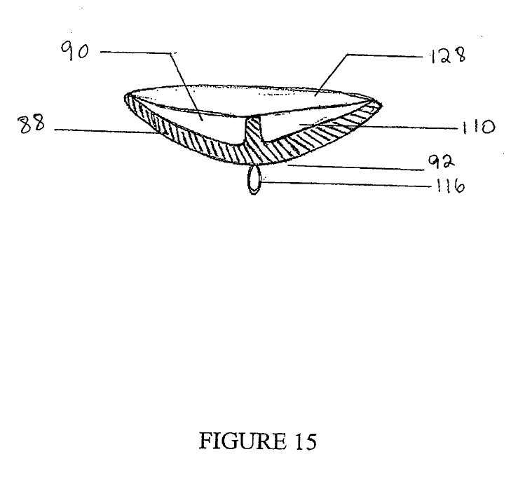

In some instances, it may not be possible to align the expandable structure 110 such that the ovary 72 or other intact organ are enclosed. An alternate form of the invention is shown in FIG. 15 in which the struts 88 expand radially outward to form a bowl-like or cup-like surface 128. The struts 88 can be straight or curved or hinged when expanded. The angle between the outer ends of the strut 88 and the central axis of the expandable structure 110 may approach about 90°. Preferably, the angle between the outer ends of the strut 88 and the central axis of the expandable structure 110 is between about 45° and about 80°. The depth of the bowl 128 defines an interior volume sufficient to capture any fluid or particles that may be formed during the surgery.

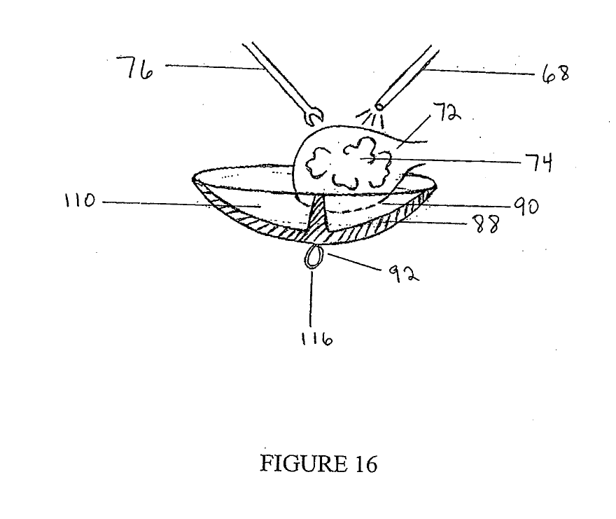

As shown in FIG. 16, in this configuration the expandable structure 110 provides a bowl-like or cup-like surface 128 positioned under the ovary 72 having a cyst 74. Similar delivery methods as described above may be used. In a preferred embodiment, the expandable structure 110 is introduced via an insertion housing 118 (not shown in FIG. 16). The cyst 74 is removed from the ovary 72 assisted by the laparoscope 68 and laparoscopic instruments 76. In this embodiment, the expandable structure 110 is used as a container to collect leakage from the surgical procedure and may partially encapsulate the ovary 72 or the bowl 128 can simply be placed below the ovary 72 or other organ or location to be treated. The ovary 72 is positioned above the expandable structure 110. The leakage from the surgical procedure is aspirated from the bowl 128 of the expandable structure. The bowl 128 is cleaned and then the expandable structure 110 is capsized and removed. The cyst tissue may be removed through the trocar sleeve (not shown). Alternatively, the cyst may be held within the expandable structure 110 and withdrawn as the capsized expandable structure 110 is withdrawn.

Preferred embodiments of the present invention allow for excision of a mass such as a cyst after the organ containing the mass, such as an ovary, has been encapsulated in the bag or expandable structure or by placing the expandable structure under the mass to be excised to provide a partial encapsulation of the mass as described above. Of course, the expandable structures described herein may be any size or shape to accommodate the mass to be removed and as determined by the nature of the particular surgery. The expandable structure need not include struts and a membrane. Any suitable expandable structure may suffice. The disclosed bag or expandable structure will allow surgical operation on the ovary without spilling the contents of the cyst into the peritoneal cavity. Consequently, there is a greatly reduced risk of peritonitis from spillage of a dermoid or mucinous cyst and also a greatly reduced risk of contamination of the peritoneal cavity with cancer cells if the cyst turns out to be malignant. Thus, a laparoscopic cystectomy can be performed regardless of the nature and the size of the cyst which is a big advantage for the patient in terms of shorter recovery time, fewer complications and fewer unnecessary oophorectomies and laparotomies. Of course, if the cyst turns out to be malignant, then additional surgery will be necessary. But if it isn't, then the patient would have benefited greatly from the laparoscopic approach. The bag or expandable structure also prevents inadvertent injury to the surrounding structures such as the bowels or the bladder and since the cyst is already deflated during the procedure, the transport of the cyst outside the abdominal cavity is also simplified for the physician.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A surgical device for enclosing an intact organ to perform a surgical procedure thereon, the device comprising:
an expandable structure having a collapsed configuration and an expanded configuration;
a membrane attached to the expandable structure; and
at least one port in the membrane adapted to allow a surgical instrument to pass therethrough to operate on the intact organ.

2. The surgical device of claim 1, wherein the membrane attached to the expandable structure is substantially funnel-shaped when the expandable structure is in its expanded configuration.

3. The surgical device of claim 1, wherein the intact organ is a live intact organ.

4. The surgical device of claim 3, wherein the intact organ is an ovary.

5. The surgical device of claim 1, wherein the membrane is adapted to be closed over a pedicle of the intact organ.

6. The surgical device of claim 1, wherein the expandable structure is self-expanding.

7. The surgical device of claim 6, wherein the expandable structure comprises a plurality of self-expanding struts.

8. A surgical device for enclosing an intact organ to perform a surgical procedure thereon, comprising:
an expandable structure comprising a plurality of struts;
a membrane attached to the struts, having an opening at a distal end thereof sufficiently large to receive the intact organ and an attachment point at a proximal end thereof adapted to manipulate and retrieve the expandable structure; and
at least one port to allow a surgical instrument to be introduced into the expandable structure to perform a surgical operation on the intact organ contained within the expandable structure.

9. The surgical device of claim 8, wherein the struts comprise spring-loaded metal prongs.

10. The surgical device of claim 8, wherein the attachment point is a flexible loop.

11. The surgical device of claim 8, wherein the intact organ is a live intact organ.

12. The surgical device of claim 8, wherein the intact organ is an ovary.

13. The surgical device of claim 8, further comprising a drawstring near the opening to close the opening.

14. The surgical device of claim 8, wherein the opening at the distal end comprises a noose-like opening.

15. The surgical device of claim 8, further comprising an insertion housing for enclosing the expandable structure.

16. The surgical device of claim 8, comprising a plurality of ports to allow surgical instruments to be introduced into the expandable structure.

17. The surgical device of claim 8, wherein the struts are self-expanding.

18. A method of performing a cystectomy, comprising:
introducing an expandable structure comprising a frame attached to a membrane capable of having a collapsed configuration and an expanded configuration into an abdominal cavity of a patient;
expanding the frame attached to the membrane to the expanded configuration to provide an opening at the distal end of the frame sufficiently large to encapsulate an ovary which is attached to the body;
positioning the frame over the ovary to encapsulate the ovary;
closing the opening of the expandable structure over an ovarian pedicle; and
introducing one or more instruments into the expandable structure to separate a cyst from the encapsulated ovary.

19. The method of claim 18, further comprising removing the expandable structure from the ovarian pedicle and releasing the ovary from the expandable structure.

20. The method of claim 18, further comprising re-closing the opening of the expandable structure with the ovary outside the expandable structure and with the removed cyst inside the expandable structure.

21. The method of claim 20, further comprising transporting the separated cyst outside of the abdominal cavity within the expandable structure.

22. The method of claim 18, wherein introducing the expandable structure into the abdominal cavity comprises inserting the expandable structure into the abdominal cavity within an insertion housing.

23. The method of claim 18, wherein the expandable structure is opened sufficiently to allow for the performance of the cystectomy inside the expandable structure.

24. The method of claim 18, wherein closing the opening of the expandable structure over the pedicle comprises pulling a drawstring closed over the ovarian pedicle.

25. The method of claim 18, wherein introducing one or more instruments into the expandable structure comprises inserting said instruments through ports in a surface of the membrane.

26. A surgical device for performing a surgical procedure, comprising:
an expandable structure comprising a plurality of struts; and
a membrane attached to the struts, having an opening at a distal end thereof and an attachment point at a proximal end thereof adapted to manipulate and retrieve the expandable structure,
wherein the expandable structure in an expanded configuration forms a bowl to collect leakage from the surgical procedure.

27. The surgical device of claim 26, wherein the struts comprise spring-loaded metal prongs.

28. The surgical device of claim 26, wherein the attachment point is a flexible loop.

29. The surgical device of claim 26, further comprising an insertion housing for enclosing the expandable structure.

30. The surgical device of claim 26, wherein the struts are self-expanding.

31. A method of performing a cystectomy, comprising:
introducing an expandable structure capable of having a collapsed configuration and an expanded configuration into an abdominal cavity of a patient;
expanding the frame attached to the membrane to the expanded configuration to provide a bowl;
positioning the bowl underneath an ovary to collect leakage from the cystectomy;
separating a cyst from the ovary; and
suctioning the leakage from the bowl.

32. The method of claim 31, wherein introducing the expandable structure into the abdominal cavity comprises inserting the expandable structure into the abdominal cavity within an insertion housing.

33. The method of claim 31, wherein the expandable structure comprises a frame attached to a membrane.
